(19) United States
(12) Patent Application Publication
(10) Pub. No.: US 2020/0138576 A1
(43) Pub. Date: May 7, 2020

(54) PERCUTANEOUS PAPILLARY MUSCLES DISPLACEMENT SYSTEM

(71) Applicant: Edwards Lifesciences Corporation, Irvine, CA (US)

(72) Inventor: Ilan Tamir, Irvine, CA (US)

(21) Appl. No.: 16/183,480

(22) Filed: Nov. 7, 2018

(51) Int. Cl. A61F 2/24

(52) U.S. Cl. CPC A61F 2/2442; A61F 2220/0083

(57) ABSTRACT

A method for treating a heart valve comprises delivering a repositioning device to a ventricle of a heart. The repositioning device comprises an anchoring element and a band. The method further comprises securing a first end of the band to the anchoring element, extending a second end of the band to encircle and at least partially contact a first papillary muscle, tightening the band to cause repositioning of at least a portion of the first papillary muscle, and locking the band in place.

### BACKGROUND

### Field

The present disclosure generally relates to the field of valve correction.

### Description of Related Art

Heart valve dysfunction can result in regurgitation and other complications due to valve prolapse from failure of valve leaflets to properly coapt. For atrioventricular valves, papillary muscle position can affect the ability of valve leaflets to function properly.

### SUMMARY

In certain embodiments, the present disclosure relates to a method for treating a heart valve. The method comprises delivering a repositioning device to a ventricle of a heart. In some embodiments, the repositioning device comprises an anchoring element and a band. The method further involves securing a first end of the band to the anchoring element, extending a second end of the band to encircle and at least partially contact a first papillary muscle, tightening the band to cause repositioning of at least a portion of the first papillary muscle, and locking the band in place.

The method may further comprise securing the second end of the band to the anchoring element. In some embodiments, tightening the band causes repositioning of the at least a portion of the first papillary muscle towards the anchoring element. Securing the second end of the band to the anchoring element may comprise passing the second end of the band through a cavity in the anchoring element.

In some embodiments, the method further comprises attaching the second end of the band to a midsection of the band. Tightening the band may cause repositioning of the at least a portion of the first papillary muscle towards a second papillary muscle. In some embodiments, the second end of the band comprises an attachment device configured to attach to a midsection of the band. The attachment device may comprise one or more of a loop and a hook. In some embodiments, attaching the second end of the band to a midsection of the band comprises tying the second end of the band to a midsection of the band. The method may further comprise extending the second end of the band to encircle and at least partially contact a second papillary muscle. In some embodiments, the second end of the band is slidably attached to a midsection of the band to allow the second end of the band to slide along the band when the band is tightened.

The first portion of cardiac tissue may be a septum of the heart, an apex region of the heart, or a papillary muscle. In some embodiments, the band has a hollow structure that is configured to fit around a guidewire.

In certain embodiments, the present disclosure relates to a cardiac repositioning device comprising an anchoring element configured to be attached to a portion of cardiac tissue and a band configured to be secured to the anchoring element and encircle and at least partially contact a first papillary muscle to reposition the first papillary muscle.

In some embodiments, the band comprises a first end and a second end, wherein each of the first end and the second end is configured to be secured to the anchoring element. The band may be further configured to be tightened to cause repositioning of at least a portion of the first papillary muscle towards the anchoring element.

The band may comprise a first end and a second end, wherein the first end is configured to be secured to the anchoring element and the second end is configured to attach to a midsection of the band. In some embodiments, the band is further configured to be tightened to cause repositioning of at least a portion of the first papillary muscle towards a second papillary muscle.

In certain embodiments, the present disclosure relates to a cardiac repositioning device comprising a first means configured to be attached to a portion of cardiac tissue and a second means configured to attach to the first means and encircle and at least partially contact a first papillary muscle to reposition the first papillary muscle.

The second means may comprise a first end and a second end, wherein each of the first end and the second end is configured to be secured to the first means. In some embodiments, the second means is further configured to be tightened to cause repositioning of at least a portion of the first papillary muscle towards the first means.

In some embodiments, the second means comprises a first end and a second end, wherein the first end is configured to be secured to the first means and the second end is configured to attach to the second means. The second means may be further configured to be tightened to cause repositioning of at least a portion of the first papillary muscle towards a second papillary muscle.

### BRIEF DESCRIPTION OF THE DRAWINGS

Various embodiments are depicted in the accompanying drawings for illustrative purposes, and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 provides a cross-sectional view of a human heart.

FIG. 2 provides a cross-sectional view of the left ventricle and left atrium of an example heart.

FIG. 3 provides a cross-sectional view of a heart experiencing mitral regurgitation.

FIG. 4 illustrates a cross-section of a heart having a single-point tension device disposed therein according to one or more embodiments.

FIG. 5 illustrates a cross-section of a heart having a multi-point tension device anchored to the septum therein according to one or more embodiments.

FIG. 6 illustrates a cross-section of a heart having a multi-point tension device anchored to an apex portion therein according to one or more embodiments.

FIG. 7 is a flow diagram illustrating a process for repositioning portions of cardiac tissue according to one or more embodiments.

### DETAILED DESCRIPTION

The headings provided herein are for convenience only and do not necessarily affect the scope or meaning of the claimed invention.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Overview

In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

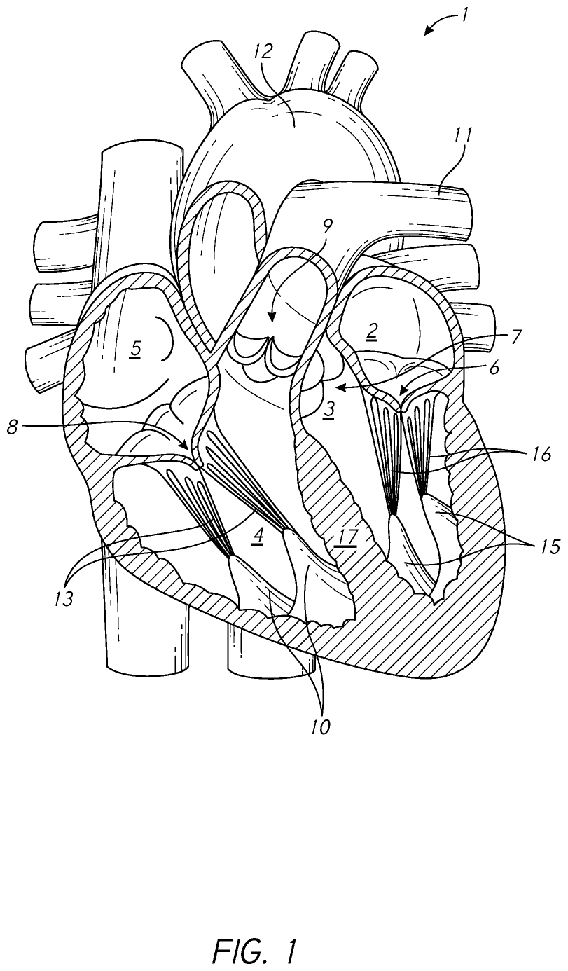

FIG. 1 illustrates an example representation of a heart 1 having various features relevant to certain embodiments of the present inventive disclosure. The heart 1 includes four chambers, namely the left atrium 2, the left ventricle 3, the right ventricle 4, and the right atrium 5. A wall of muscle 17, referred to as the septum, separates the left 2 and right 5 atria and the left 3 and right 4 ventricles. The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (i.e., systole) and open during ventricular expansion (i.e., diastole). The valves of the heart 1 further include the pulmonary valve 9, which separates the right ventricle 4 from the pulmonary artery 11, and may be configured to open during systole so that blood may be pumped toward the lungs, and close during diastole to prevent blood from leaking back into the heart 1 from the pulmonary artery 11. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

Heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant, and press back against the leaflets. As a result, the leaflets/cusps come in apposition to each other, thereby closing the flow passage.

The atrioventricular (i.e., mitral 6 and tricuspid 8) heart valves may further comprise a collection of chordae tendineae and papillary muscles for securing the leaflets of the respective valves to promote and/or facilitate proper coaptation of the valve leaflets and prevent prolapse thereof. The papillary muscles, for example, may generally comprise finger-like projections from the ventricle wall. With respect to the tricuspid valve 8, the normal tricuspid valve may comprise three leaflets (two shown in FIG. 1) and three corresponding papillary muscles 10 (two shown in FIG. 1). The leaflets of the tricuspid valve 8 may be referred to as the anterior, posterior and septal leaflets, respectively. The valve leaflets are connected to the papillary muscles 10 by the chordae tendineae 13, which are disposed in the right ventricle 4 along with the papillary muscles 10. Although tricuspid valves are described herein as comprising three leaflets, it should be understood that tricuspid valves may occur with two or four leaflets in certain patients and/or conditions; the principles relating to papillary muscle repositioning disclosed herein are applicable to atrioventricular valves having any number of leaflets and/or papillary muscles associated therewith.

The right ventricular papillary muscles 10 originate in the right ventricle wall, and attach to one or more of the anterior, posterior and septal leaflets of the tricuspid valve via the right ventricle chordae tendineae 13. The right ventricle papillary muscles 10 may have variable anatomy; the anterior papillary may generally be the most prominent of the papillary muscles. The right ventricle papillary muscles 10 may serve to secure the leaflets of the tricuspid valve 8 to prevent prolapsing of the leaflets into the right atrium 5 during ventricular systole. Tricuspid regurgitation can be the result of papillary dysfunction or chordae rupture.

With respect to the mitral valve 6, a normal mitral valve may comprise two leaflets (anterior and posterior) and two corresponding papillary muscles 15. The papillary muscles 15 originate in the left ventricle wall and project into the left ventricle 3. Generally, the anterior leaflet may cover approximately two-thirds of the valve annulus. Although the anterior leaflet covers a greater portion of the annulus, the posterior leaflet may comprise a larger surface area in certain anatomies.

The valve leaflets of the mitral valve 6 may be prevented from prolapsing into the left atrium 2 by the action of the left ventricle chordae tendineae 16 tendons connecting the valve leaflets to the left ventricle papillary muscles 15. The relatively inelastic chordae tendineae are attached at one end to the papillary muscles and at the other to the valve leaflets; left ventricle chordae tendineae 16 from each of the left ventricle papillary muscles 15 are attached to a respective leaflet of the mitral valve 6. Thus, when the left ventricle 3 contracts, the intraventricular pressure forces the valve to close, while the left ventricle chordae tendineae 16 keep the leaflets coapting together and prevent the valve from opening in the wrong direction, thereby preventing blood to flow back to the left atrium 2. The various chords of the chordae tendineae may have different thicknesses, wherein relatively thinner chords are attached to the free leaflet margin, while relatively thicker chords (e.g., strut chords) are attached farther away from the free margin.

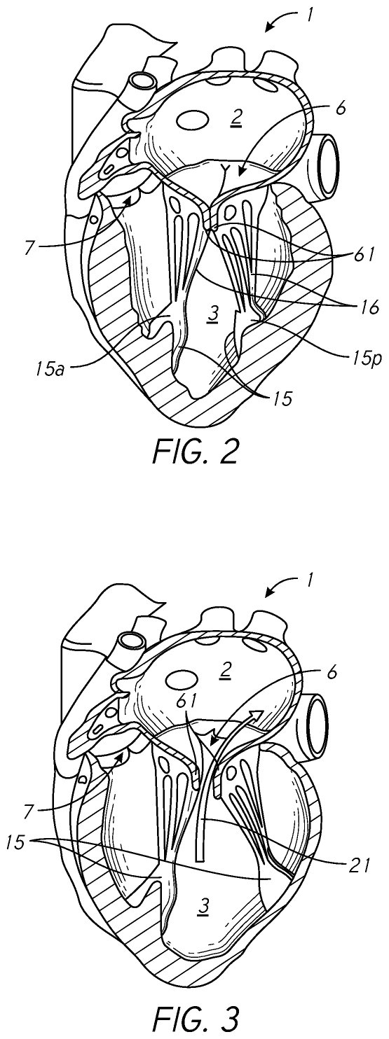

FIG. 2 provides a cross-sectional view of the left ventricle 3 and left atrium 2 of an example heart 1. The diagram of FIG. 2 shows the mitral valve 6, wherein the disposition of the valve 6, papillary muscles 15 and/or chordae tendineae 16 may be illustrative as providing for proper coapting of the valve leaflets to advantageously at least partially prevent regurgitation and/or undesirable flow into the left atrium from the left ventricle 3 and vice versa. Although a mitral valve 6 is shown in FIG. 2 and various other figures provided herewith and described herein in the context of certain embodiments of the present disclosure, it should be understood that papillary muscle repositioning principles disclosed herein may be applicable with respect to any atrioventricular valve and associated anatomy (e.g., papillary muscles, chordae tendineae, ventricle wall, etc.), such as the tricuspid valve.

As described above, with respect to a healthy heart valve as shown in FIG. 2, the valve leaflets 61 may extend inward from the valve annulus and come together in the flow orifice to permit flow in the outflow direction (e.g., the downward direction in FIG. 2) and prevent backflow or regurgitation toward the inflow direction (e.g., the upward direction in FIG. 2). For example, during atrial systole, blood flows from the atria 2 to the ventricle 3 down the pressure gradient, resulting in the chordae tendineae 16 being relaxed due to the atrioventricular valve 6 being forced open. When the ventricle 3 contracts during ventricular systole, the increased blood pressures in both chambers may push the valve 6 closed, preventing backflow of blood into the atria 2. Due to the lower blood pressure in the atria compared to the ventricles, the valve leaflets may tend to be drawn toward the atria. The chordae tendineae 16 can serve to tether the leaflets and hold them in a closed position when they become tense during ventricular systole. The papillary muscles 15 provide structures in the ventricles for securing the chordae tendineae 16 and therefore allowing the chordae tendineae 16 to hold the leaflets in a closed position. The papillary muscles 15 may include a first papillary muscle 15a (e.g., an anterolateral papillary muscle, which may be primarily tethered to the anterior leaflet, for example) and a second papillary muscle 15p (e.g., the posteromedial papillary muscle, which may be primarily tethered to the posterior leaflet, for example). Each of the first papillary muscle 15a and second papillary muscle 15p may provide chordae tendinae 16 to each valve leaflet (e.g., the anterior and posterior leaflets). With respect to the state of the heart 1 shown in FIG. 2, the proper coaptation of the valve leaflets, which may be due in part to proper position of the papillary muscles 15, may advantageously result in mitral valve operation substantially free of leakage.

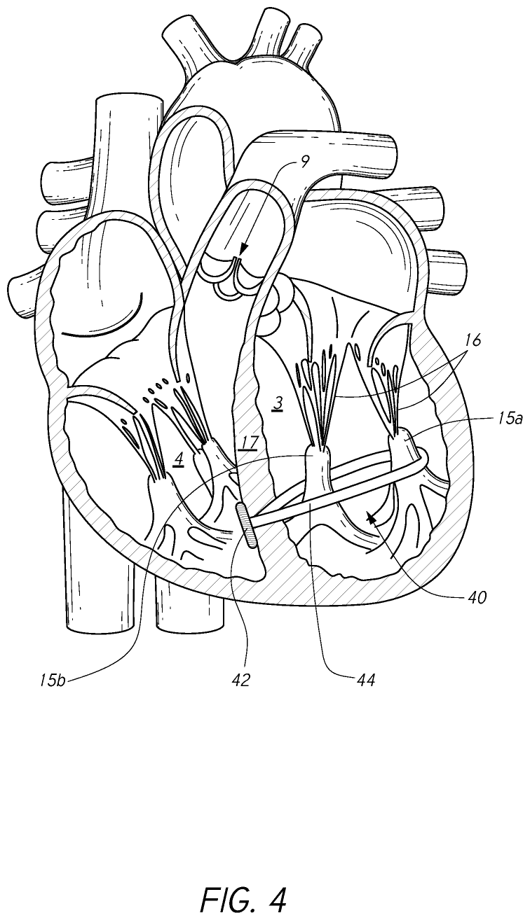

Heart valve disease represents a condition in which one or more of the valves of the heart fails to function properly. Diseased heart valves may be categorized as stenotic, wherein the valve does not open sufficiently to allow adequate forward flow of blood through the valve, and/or incompetent, wherein the valve does not close completely, causing excessive backward flow of blood through the valve when the valve is closed. In certain conditions, valve disease can be severely debilitating and even fatal if left untreated. With regard to incompetent heart valves, over time and/or due to various physiological conditions, the position of papillary muscles may become altered, thereby potentially contributing to valve regurgitation. For example, as shown in FIG. 3, which illustrates a cross-sectional view of a heart 1 experiencing mitral regurgitation flow 21, dilation of the left ventricle may cause changes in the position of the papillary muscles 15 that allow flow 21 back from the ventricle 3 to the atrium 2. Dilation of the left ventricle can be caused by any number of conditions, such as focal myocardial infarction, global ischemia of the myocardial tissue, or idiopathic dilated cardiomyopathy, resulting in alterations in the geometric relationship between papillary muscles and other components associated with the valve(s) that can cause valve regurgitation. Functional regurgitation may further be present even where the valve components may be normal pathologically, yet may be unable to function properly due to changes in the surrounding environment. Examples of such changes include geometric alterations of one or more heart chambers and/or decreases in myocardial contractility. In any case, the resultant volume overload that exists as a result of an insufficient valve may increase chamber wall stress, which may eventually result in a dilatory effect that causes papillary muscle alteration resulting in valve dysfunction and degraded cardiac efficiency.

With further reference to FIG. 3, the heart 1 is shown in a state where functional mitral valve regurgitation (FMR) is present. FMR may be considered a disease of the left ventricle 3, rather than of the mitral valve 6. For example, mitral valve regurgitation may occur when the left ventricle 3 of the heart 1 is distorted or dilated, displacing the papillary muscles 15 that support the two valve leaflets 61. The valve leaflets 61 therefore may no longer come together sufficiently to close the annulus and prevent blood flow back into the atrium 2. If left untreated, the FMR experienced in the state shown in FIG. 3 may overload the heart 1 and can possibly lead to or accelerate heart failure. Solutions presented herein provide devices and methods for moving the papillary muscles 15 closer to their previous position, which may advantageously reduce the occurrence of mitral regurgitation.

As shown in FIG. 3, the leaflets 61 of the mitral valve (or tricuspid valve) are not in a state of coaptation, resulting in an opening between the mitral valve leaflets 61 during the systolic phase of the cardiac cycle, which allows the leakage flow 21 of fluid back up into the atrium 2. The papillary muscles 15 may be displaced due to dilation of the left ventricle 3, or due to one or more other conditions, as described above, which may contribute to the failure of the valve 6 to close properly. The failure of the valve leaflets 61 to coapt properly may result in unwanted flow in the outflow direction (e.g., the upward direction in FIG. 3) and/or unwanted backflow or regurgitation toward the inflow direction (e.g., the downward direction in FIG. 2).

Certain embodiments disclosed herein provide solutions for incompetent heart valves that involve ventricular wall and/or papillary muscle repositioning. Solutions presented herein may be used to at least partially change the position of one or more papillary muscles and/or ventricular walls in order to reduce the occurrences and/or severity of regurgitation, such as mitral regurgitation. Mitral valve regurgitation often may be driven by the functional/physical positioning changes described above, which may cause papillary muscle displacement and/or dilatation of the valve annulus. As the papillary muscles move away from the valve annulus, the chordae connecting the muscles to the leaflets may become tethered. Such tethering may restrict the leaflets from closing together, either symmetrically or asymmetrically, depending on the relative degree of displacement between the papillary muscles. Moreover, as the annulus dilates in response to chamber enlargement and increased wall stress, increases in annular area and changes in annular shape may increase the degree of valve insufficiency.

Various techniques that suffer from certain drawbacks may be implemented for treating mitral valve dysfunction, including surgical repair or replacement of the diseased valve or medical management of the patient, which may be appropriate/effective primarily in early stages of mitral valve dysfunction, during which levels of regurgitation may be relatively low. For example, such medical management may generally focus on volume reductions, such as diuresis or afterload reducers, such as vasodilators, for example. Valve replacement operations may also be used to treat regurgitation from valve dysfunction. However, such operations can result in ventricular dysfunction or failure following surgery. Further limitations to valve replacement solutions may include the potential need for lifelong therapy with powerful anticoagulants in order to mitigate the thromboembolic potential of prosthetic valve implants. Moreover, in the case of biologically-derived devices, such as those used as mitral valve replacements, the long-term durability may be limited. Another commonly employed repair technique involves the use of annuloplasty rings to improve mitral valve function. An annuloplasty may be placed in the valve annulus and the tissue of the annulus sewn or otherwise secured to the ring. Annuloplasty rings can provide a reduction in the annular circumference and/or an increase in the leaflet coaptation area. However, annuloplasty rings may flatten the saddle-like shape of the valve and/or hinder the natural contraction of the valve annulus. In addition, various surgical techniques may be used to treat valve dysfunction. However, such techniques may suffer from various limitations, such as requiring opening the heart to gain direct access to the valve and the valve annulus. Therefore, cardiopulmonary bypass may be required, which may introduce additional morbidity and mortality to the surgical procedures. Additionally, for surgical procedures, it can be difficult or impossible to evaluate the efficacy of the repair prior to the conclusion of the operation.

Disclosed herein are devices and methods for treating valve dysfunction without the need for cardiopulmonary bypass and without requiring major remodeling of the dysfunctional valve. In particular, passive techniques to lower ventricular volume and/or change the shape and/or position of the papillary muscles are disclosed for improving ventricular function and/or reducing regurgitation while maintaining substantially normal leaflet anatomy. Further, various embodiments disclosed herein provide for the treatment of valve dysfunction that can be executed on a beating heart, thereby allowing for the ability to assess the efficacy of the ventricular remodeling and/or papillary muscle repositioning treatment and potentially implement modification thereto without the need for bypass support.

Some embodiments involve encircling, wrapping around, hooking, or otherwise engaging one or more papillary muscles to bring at least portions of the papillary muscles closer together and/or towards an anchoring point. Papillary muscles may be engaged through use of a band configured to at least partially wrap around one or more papillary muscles. The term “band” is used herein according to its broad and ordinary meaning and may refer to any tube, suture, string, cord, wire, or other length of material. In some embodiments, the band may be hollow to allow a guidewire or similar device to pass through the band.

In certain embodiments, the band may be configured to form a closed loop (e.g., a ring) that may be fitted around one or more papillary muscles. For example, with reference to FIG. 2, the band may be delivered to the left ventricle 3 and a first end of the band may be wrapped around the anterolateral 15a and posteromedial 15p papillary muscles. A first end of the band may form a complete loop around the papillary muscles and connect to a second end of the band, a midsection of the band, and/or to a connection device. The loop may be tightened until a desired amount of pressure is applied to one or more of the papillary muscles to cause at least portions of the papillary muscles to move closer together. Multiple ends of the band may be tied together and/or secured to a connection device and/or locking device to maintain tightness of the loop.

In some embodiments, the band may be secured and/or anchored to one or more anchoring elements that are engaged with portions of cardiac tissue, for example the septum, a lateral wall, the apex region, and/or the papillary muscles. One or multiple ends of the band may be anchored to the anchoring element. In one use case, a band having two ends wraps around the papillary muscles and both ends are anchored to a common anchoring element. In another use case, a band having two ends wraps around the papillary muscles and a first end is anchored to an anchoring element while a second end attaches to a midsection (e.g., a portion between the first end and a second end) of the band. By anchoring to an anchoring element, the band may be more effectively prevented from moving up or down along the papillary muscles after it is locked in place. Movement of the band along the papillary muscles may result in the band contacting chordae tendinae attached to the papillary muscles, potentially causing abrasion at the chordae tendinae, or moving towards the bases of the papillary muscles where the band may be less effective in remodeling the papillary muscles. The anchoring element may be anchored at a region of cardiac tissue to secure the band in a desired position. For example, the anchoring element may be anchored at a portion of the septum such that a band extending from the anchoring element is held at a point on a papillary muscle that is sufficiently distal from the chordae tendinae and/or base of the papillary muscle.

In some embodiments, the band and/or anchoring element can be delivered and adjusted using a transfemoral (artery), transapical, or transseptal procedure. Once in place, the band and/or the anchoring element can be detached from the delivery system and left in the heart an implant. Some embodiments do not require puncturing the papillary muscles.

Some devices may include multiple anchors. For example, one anchor may be attached at the septal wall and another anchor may be attached at an apex region. In another example, a first anchor may be attached at or near a base of a first papillary muscle and a second anchor may be attached at or near a base of a second papillary muscle.

### Papillary Muscle Repositioning Devices

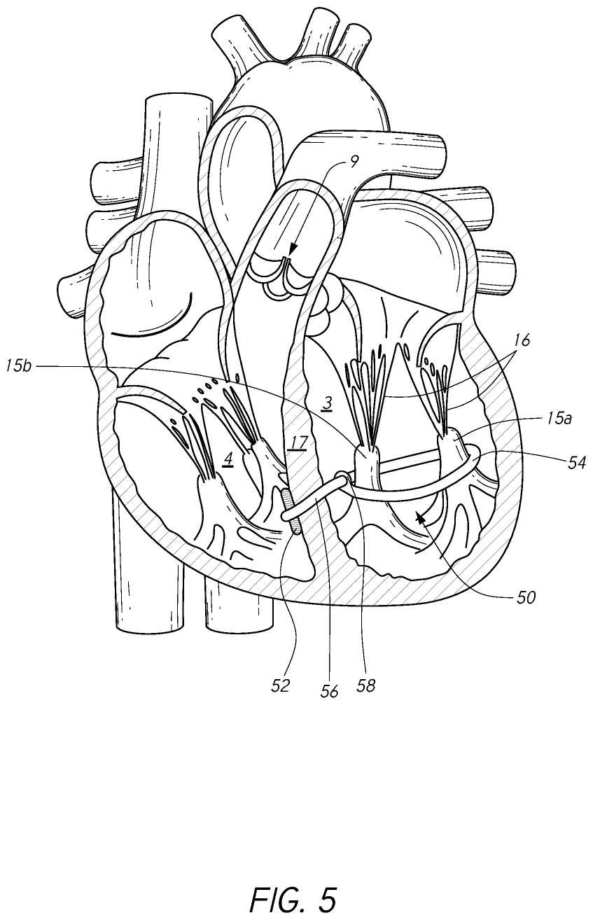

FIGS. 4-6 illustrate a cross-section of a heart 1 showing a left ventricle 3 thereof. Although certain disclosure herein is presented in the context of the left ventricle and associated anatomy (e.g., valves, papillary muscles, chordae tendineae, ventricle wall, etc.), it should be understood that the principles disclosed herein may be applicable in any ventricle of the heart (e.g., right ventricle) and associated anatomy (e.g., tricuspid valve, papillary muscles, chordae tendineae, ventricle wall, etc.). As described above, in a normal heart, the papillary muscles may contract during the heart cycle to assist in maintaining proper valve function. Reductions in, or failure of, the papillary muscle function can contribute to valve dysfunction and/or regurgitation, which may be caused by infarction at or near the papillary muscle, ischemia, or other causes, such as idiopathic dilated cardiomyopathy, for example.

### Single-Point Tension Device

FIG. 4 shows a single-point tension device 40, which may be implanted in the left ventricle 3 (or right ventricle in another embodiment) to at least partially reposition one or more papillary muscles. The single-point tension device 40 may pull a first papillary muscle 15a and/or a second papillary muscle 15b towards an anchoring point of the single-point tension device 40 (e.g., towards the anchoring element 42). By repositioning one or more of the papillary muscles towards the anchoring point of the single-point tension device 40, the traction of the chordae tendineae 16 on the corresponding leaflet of the mitral valve may be lessened, thereby resulting in improved coaptation of the mitral valve leaflets during closure of the valve. In certain conditions/patients, moving the first papillary muscle 15a and the second papillary muscle 15b towards the anchoring point of the single-point tension device 40 may help correct mitral valve insufficiency due to dysfunction or rupture of the papillary muscles.

In some embodiments, the single-point tension device 40 may comprise an anchoring element 42 and a band 44. The band 44 may wrap around one or more papillary muscles and both ends of the band 44 may be anchored at the anchoring element 42 to create a single tension point at the anchoring element 42. The single-point tension device 40, when placed into the ventricle, may form a “droplet” shape. The anchoring element 42 may be situated at a portion of the myocardium, for example the septum 17 in the example shown in FIG. 4. In some embodiments, the anchoring point for the anchoring element 42 may be the septum 17, lateral wall, or apex region. The band 44 may pass through at least a portion of the septum 17, lateral wall, posterior wall, and/or apex to attach to, pass through, and/or extend from the anchoring element 42. With both ends of the band 44 connected to the anchoring element 42, the single-point tension device 40 may tend to pull the papillary muscles towards the anchoring element 42. In some embodiments, the single-point tension device 40 may apply a pulling force to only one of the papillary muscles (e.g., the papillary muscle situated further from the anchoring element 42).

With respect to embodiments in which the single-point tension device 40 is implanted in the right ventricle, the device may serve to correct tricuspid regurgitation, which, similar to mitral regurgitation, involves a disorder in which the tricuspid valve does not close tightly enough to prevent backflow through the valve. During tricuspid regurgitation, blood may flow backward into the right atrium when the right ventricle contracts. Such tricuspid valve dysfunction may result from the increase in size of the right ventricle. For example, enlargement or dilation of the right ventricle may result from high blood pressure in the arteries of the lungs, or from other heart problems, such as poor squeezing of the left side of the heart, or from problems with the opening or closing of another one of the heart valves.

The single-point tension device 40 may be inserted non-surgically in, for example, a transcatheter procedure (e.g., transfemoral, transseptal, transapical, etc.), wherein the single-point tension device 40 may be inserted, for example, into the left ventricle 3 from the aorta 12 through the aortic valve 7 and the anchoring element 42 may be anchored into and/or passed through the septum 17 to be situated on the right ventricle 4 side of the septum 17. In some embodiments, the single-point tension device 40 may be inserted into the right ventricle 4 from the pulmonary artery through the pulmonary valve 9 and the anchoring element 42 may be anchored into and/or passed through the septum 17 to be situated on the left ventricle 3 side of the septum 17.

The anchoring element 42 may comprise one or more corkscrews, barbs, balloons, hooks, and/or any other mechanisms suitable for anchoring into the septum 17 or other tissue wall. In some embodiments, the anchoring element 42 may comprise a portion configured to pass through the tissue wall and expand on a side of the tissue wall that is distal from the entry point to lock the anchoring element 42 in place. For example, the anchoring element 42 may comprise a substantially flat pad (e.g., a pledget) in a form similar to an Amplatzer™ or other similar device. The pad may be configured to lay substantially flat against, or relative to a guidewire and/or the band 44 during insertion through the tissue wall, and after exiting the tissue wall may extend perpendicularly from the proximal portion of the guidewire and/or band 44, and further lay substantially flat against the distal side surface of the tissue wall. In an embodiment, the pad may be at least partially composed of felt or a similar material. In some embodiments, the anchoring element 42 may be anchored to a papillary muscle, for example at a base region of a papillary muscle. In certain embodiments, the single-point tension device 40 may comprise multiple anchoring elements 42.

In some embodiments, the anchoring element 42 may comprise a wire (e.g., composed of Nitinol or similar material) that is shape-set in a ring or other shape. The anchoring element 42 may be collapsible to fit into a catheter and, after exiting the catheter, may return to its shape-set form (e.g., a ring) and may be positioned to lay substantially flat against a tissue wall. In some embodiments, the anchoring element 42 may comprise a cloth and/or Nitinol mesh which may be configured to attach to a midsection of the band 44.

The anchoring element 42 may be delivered to any point on the septum 17 or other tissue wall. The position of the anchoring element 42 on the septum 17 or other tissue wall may be chosen based on a desired contact point of the single-point tension device 40 at a first papillary muscle 15a and/or second papillary muscle 15b. For example, it may be desirable to wrap the band 44 around a portion of the first papillary muscle 15a and/or second papillary muscle 15b that is sufficiently distal from the chordae 16 to prevent contact with and/or abrasion on the chordae tendinae 16. Accordingly, the anchoring element 42 may be positioned lower on the septum 17 such that the anchoring element 42 does not apply an upward pulling force on the band 44 towards the chordae tendinae 16. Moreover, in some cases it may be desirable to wrap the band 44 around a base portion of the first papillary muscle 15a and/or second papillary muscle 15b to cause greater ventricle remodeling and/or improved durability, while in other cases it may be desirable to wrap the band 44 around a middle portion of the first papillary muscle 15a and/or second papillary muscle 15b to cause greater papillary muscle remodeling. In each case, the position of the anchoring element 42 may be chosen to support the desired engagement point(s) on the one or more papillary muscles.

The band 44 may be a tube, suture, string, cord, wire, or similar device and may be composed of plastic, metal, Nitinol, Teflon, polymer, or other material. In some embodiments, the band 44 may be formed using a laser-cutting procedure. The band 44 may have relatively high compliance to maintain a desired amount of force against the papillary muscles after the band 44 is tightened, though the band 44 may be at least partially elastic to allow for some amount of stretching. In some embodiments, the band 44 may have a hollow structure to allow a guidewire or similar to device to pass through it during delivery of the band 44. For example, a guidewire may first be inserted into a ventricle and wrapped around the papillary muscles. After the guidewire is in place, the band 44 may be fed over the guidewire to similarly surround the papillary muscles. After the band 44 is in place, the guidewire may be removed via a transcatheter procedure. In certain embodiments, the band 44 may be delivered without use of a guidewire.

One or more ends of the band 44 may be attached to the anchoring element 42. In some embodiments, one or more ends of the band 44 may be pre-attached to the anchoring element 42 at delivery of the anchoring element 42 and/or one or more ends of the band 44 may be attached to the anchoring element 42 after delivery of the anchoring element 42. The band 44 may attach to the anchoring element 42 through use of various attachment mechanisms.

In some embodiments, the anchoring element 42 may be positioned in a first ventricle (e.g., the right ventricle 4 in FIG. 4) while the band 44 encircles papillary muscles in a second ventricle (e.g., the left ventricle 3 in FIG. 4). Accordingly, in order to attach to the anchoring element 42, at least a portion of the band 44 may pass through and/or be embedded in the septum 17 and/or other tissue wall. In some embodiments, multiple ends of the band 44 may pass through a common hole and/or passageway in the septum 17 and/or other tissue wall

The band 44 may contact one or more papillary muscles. In some embodiments, the band 44 may encircle multiple papillary muscles but may contact only one or two of the papillary muscles. For example, the band 44 may wrap around and contact the first papillary muscle 15a but may not contact the second papillary muscle 15b while encircling both papillary muscles 15a, 15b. In some embodiments, the band 44 may apply disparate amounts of force to different papillary muscles. The disparate amounts of force may be based at least in part on the relative distances of the papillary muscles from the anchoring element 42. For example, the band 44 may apply a greater force to the first papillary muscle 15a than to the second papillary muscle 15b because the first papillary muscle 15a may be further from the anchoring element 42 than the second papillary muscle 15b. Accordingly, the single-point tension device 40 may cause different amounts of remodeling to different papillary muscles. While in some embodiments the band 44 may wrap around one or more papillary muscles, the band 44 may additionally or alternatively pierce the one or more papillary muscles.

In some embodiments, the anchoring element 42 may comprise a locking mechanism to lock the band 44 in place in order to maintain a desired amount of pressure on the papillary muscles. The locking mechanism may prevent movement of the band 44 through the anchoring element 42 in a single direction or multiple directions. For example, after wrapping around and/or engaging the papillary muscles, the band 44 may be delivered through the anchoring element 42 (e.g., in a direction that is away from the papillary muscles. A surgeon may continue to pull the band 44 through the anchoring element 42 to tighten the band 44 and increase an amount of pressure on the papillary muscles. Accordingly, the locking mechanism may allow movement of the band 44 through the anchoring element 42 in the direction that is away from the papillary muscles while preventing movement in the opposite direction to prevent loosening of the band 44. After the single-point tension device 40 is locked in the desired position, excess length of the band 44 may be removed.

### Multi-Point Tension Device

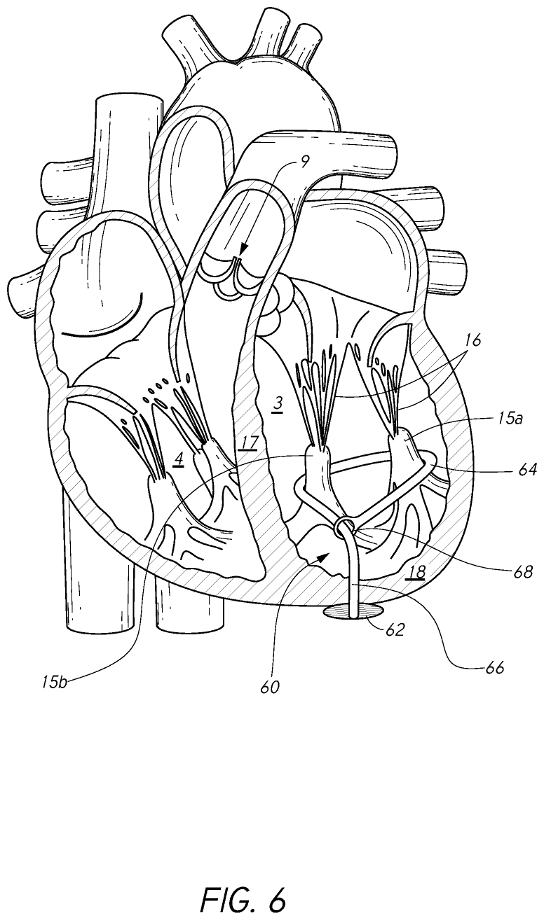

FIG. 5 shows a multi-point tension device 50 anchored to the septum 17 and FIG. 6 shows a multi-point tension device 60 anchored at or near an apex 18 region of the heart 1. The multi-point tension device 50, 60 may be implanted in the left ventricle 3 (or right ventricle in another embodiment) to at least partially pull one or more papillary muscles towards each other. That is, the multi-point tension device 50, 60 may pull a first papillary muscle 15a towards a second papillary muscle 15b and/or the multi-point tension device 50, 60 may pull the second papillary muscle 15b towards the first papillary muscle 15a, which may cause one or both of the papillary muscles to reposition towards a space between the papillary muscles.

In some embodiments, the multi-point tension device 50, 60 may comprise an anchoring element 52, 62 and a band 54, 64. In certain embodiments, a first end 56, 66 of the band 54, 64 is connected to the anchoring element 52, 62 at the myocardium and a second end 58, 68 of the band 54, 64 is connected to a midsection (e.g., between the first end 56, 66 and the second end 58, 68) of the band 54, 64 to form a closed loop that is set apart from the anchoring element 52, 62 to form a “lasso” shape. In this way, the multi-point tension device 50, 60 may cause tension at both the anchoring point of the anchoring element 52, 62 and the connection point of the second end 58, 68. In some embodiments, the second end 58, 68 may comprise a connecting mechanism (e.g., a hook or loop) that can at least partially wrap around, pinch, or otherwise attach to and slide along the midsection of the band 54, 64. In this way, the band 54, 64 may form an adjustable loop around one or more papillary muscles 15a, 15b that can be tightened or loosened in a controlled manner.

The anchoring element 52, 62 may be situated at a portion of the myocardium, for example the septum 17 in the example shown in FIG. 5 and/or the apex 18 in the example shown in FIG. 6. In some embodiments, the anchoring point for the anchoring element 52, 62 may be the septum 17, lateral wall, or apex 18. The band 54, 64 may pass through at least a portion of the septum 17, lateral wall, posterior wall, and/or apex 18 to attach to the anchoring element 52, 62.

When the band 54, 64 is tightened (e.g., when a surgeon pulls on the band 54, 64) the formed loop of the multi-point tension device 50, 60 may reduce in area and accordingly apply increased pressure to both the first papillary muscle 15a and the second papillary muscle 15b and cause the papillary muscles to move closer together.

The multi-point tension device 50, 60 may be inserted non-surgically in, for example, a transcatheter procedure (e.g., transfemoral, transseptal, transapical, etc.), wherein the multi-point tension device 50, 60 may be inserted, for example, into the left ventricle 3 from the aorta 12 through the aortic valve 7 and the anchoring element 52, 62 may be anchored into and/or passed through the septum 17 to be situated on the right ventricle 4 side of the septum 17. In some embodiments, the multi-point tension device 50, 60 may be inserted into the right ventricle 4 from the pulmonary artery through the pulmonary valve 9 and the anchoring element 52, 62 may be anchored into and/or passed through the septum 17 to be situated on the left ventricle 3 side of the septum 17.

The anchoring element 52, 62 may comprise one or more corkscrews, barbs, balloons, hooks, and/or any other mechanisms suitable for anchoring into the septum 17, apex 18, or other tissue wall. In some embodiments, the anchoring element 52, 62 may comprise a portion configured to pass through the tissue wall and expand on a side of the tissue wall that is distal from the entry point to lock the anchoring element 52, 62 in place. For example, the anchoring element 52, 62 may comprise a substantially flat pad (e.g., a pledget) in a form similar to an Amplatzer™ or other similar device. The pad may be configured to lay substantially flat against, or relative to a guidewire and/or the band 54, 64 during insertion through the tissue wall, and after exiting the tissue wall may extend perpendicularly from the proximal portion of the guidewire and/or band 54, 64, and further lay substantially flat against the distal side surface of the tissue wall. In an embodiment, the pad may be at least partially composed of felt or a similar material. In some embodiments, the anchoring element 52, 62 may be attached to a papillary muscle, for example at a base region of a papillary muscle. In certain embodiments, the multi-point tension device 50, 60 may comprise multiple anchoring elements 52, 62.

In some embodiments, the anchoring element 52, 62 may comprise a wire (e.g., composed of Nitinol or a similar material) that is shape-set in a ring or other shape. The anchoring element 52, 62 may be collapsible to fit into a catheter and, after exiting the catheter, may return to its shape-set form (e.g., a ring) and may be positioned to lay substantially flat against a tissue wall. In some embodiments, the anchoring element 52, 62 may comprise a cloth and/or Nitinol mesh which may be configured to attach to a midsection of the band 54, 64.

The anchoring element 52, 62 may be delivered to any point on the septum 17, apex 18, or other tissue wall. The position of the anchoring element 52, 62 on the septum 17, apex 18, or other tissue wall may be chosen based on a desired contact point of the multi-point tension device 50, 60 at a first papillary muscle 15a and/or second papillary muscle 15b. For example, it may be desirable to wrap the band 54, 64 around a portion of the first papillary muscle 15a and/or second papillary muscle 15b that is sufficiently distal from the chordae tendinae 16 to prevent contact with and/or abrasion on the chordae tendinae 16. Accordingly, for example, the anchoring element 52, 62 may be positioned lower on the septum 17 such that the anchoring element 52, 62 does not apply an upward pulling force on the band 54, 64 towards the chordae 16. Moreover, in some cases it may be desirable to wrap the band 54, 64 around a base portion of the first papillary muscle 15a and/or second papillary muscle 15b to cause greater ventricle remodeling and/or improved durability while in other cases it may be desirable to wrap the band 54, 64 around a middle portion of the first papillary muscle 15a and/or second papillary muscle 15b to cause greater papillary muscle remodeling. In each case, the position of the anchoring element 52, 62 may be chosen to support the desired engagement point(s) on the one or more papillary muscles.

The band 54, 64 may be a tube, suture, string, cord, wire, or similar device and may be composed of plastic, metal, Nitinol, Teflon, polymer, or other material. In some embodiments, the band 54, 64 may be formed using a laser-cutting procedure. The band 54, 64 may have relatively high compliance in order to maintain a desired amount of force against the papillary muscles after the band 54, 64 is tightened, though the band 54, 64 may be at least partially elastic to allow for some amount of stretching. In some embodiments, the band 54, 64 may have a hollow structure to allow a guidewire or similar to device to pass through it during delivery of the band 54, 64. For example, a guidewire may first be inserted into a ventricle and wrapped around the papillary muscles. After the guidewire is in place, the band 54, 64 may be fed over the guidewire to similarly surround the papillary muscles. After the band 54, 64 is in place, the guidewire may be removed via a transcatheter procedure. In certain embodiments, the band 54, 64 may be delivered without use of a guidewire.

One or more ends of the band 54, 64 may be attached to the anchoring element 52, 62. In some embodiments, one or more ends of the band 54, 64 may be pre-attached to the anchoring element 52, 62 at delivery of the anchoring element 52, 62 and/or one or more ends of the band 54, 64 may be attached to the anchoring element 52, 62 after delivery of the anchoring element 52, 62. The band 54, 64 may attach to the anchoring element 52, 62 through use of various attachment mechanisms.

In some embodiments, the anchoring element 52, 62 may be positioned in a first ventricle (e.g., the right ventricle 4 in FIG. 5) while the band 54, 64 encircles papillary muscles in a second ventricle (e.g., the left ventricle 3 in FIG. 5). Accordingly, in order to attach to the anchoring element 52, 62, at least a portion of the band 54, 64 may pass through and/or be embedded in the septum 17, apex 18, and/or other tissue wall. In some embodiments, multiple ends of the band 54, 64 may pass through a common hole and/or passageway in the septum 17, apex 18, and/or other tissue wall

The band 54, 64 may contact one or more papillary muscles. While in some embodiments the band 54, 64 may wrap around one or more papillary muscles, the band 54, 64 may additionally or alternatively pierce the one or more papillary muscles.

In some embodiments, the anchoring element 52, 62 may comprise a locking mechanism to lock the band 54, 64 in place in order to maintain a desired amount of pressure on the papillary muscles. The locking mechanism may prevent movement of the band 54, 64 through the anchoring element 52, 62 in a single direction or multiple directions. For example, after wrapping around and/or engaging the papillary muscles, the band 54, 64 may be delivered through the anchoring element 52, 62 (e.g., in a direction that is away from the papillary muscles). A surgeon may continue to pull the band 54, 64 through the anchoring element 52, 62 to tighten the band 54, 64 and increase an amount of pressure on the papillary muscles. Accordingly, the locking mechanism may comprise a ratchet or similar mechanism to allow movement of the band 54, 64 through the anchoring element 52, 62 in the direction that is away from the papillary muscles while preventing movement in the opposite direction to prevent loosening of the band 54, 64. After the multi-point tension device 50, 60 is locked in the desired position, excess length of the band 54, 64 may be removed.

### Papillary Muscle Repositioning Processes

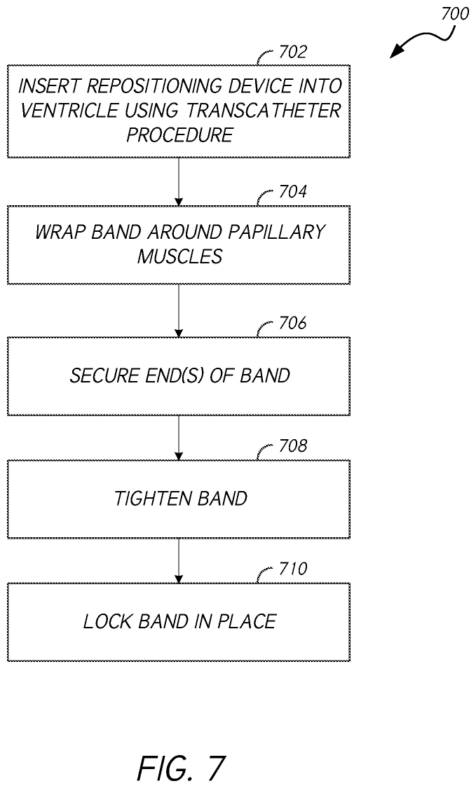

FIG. 7 is a flow diagram representing a process 700 for repositioning one or more papillary muscles and/or other anatomy of a ventricle of the heart according to one or more embodiments disclosed herein. While some steps of the process 700 may be directed to the left ventricle, such steps may also be applied to the right ventricle.

At step 702, the process 700 involves inserting a repositioning device into a ventricle of the heart using a transcatheter procedure. For example, the repositioning device may be delivered using a transfemoral, transendocardial, transcoronary, transseptal, transapical, or other approach. Alternatively, the repositioning device may be introduced into the desired location during an open-chest surgical procedure, or using other surgical or non-surgical techniques known in the art.

In some embodiments, the repositioning device may be inserted into the right ventricle where it can engage the papillary muscles in the right ventricle or may be passed through the septum into the left ventricle. Alternatively, the repositioning device may be inserted into the left ventricle where it can engage the papillary muscles in the left ventricle or may be passed through the septum into the right ventricle. For a transapical procedure, the repositioning device may be inserted through the apex via a catheter.

The repositioning device may comprise one or more connected and/or connectable elements. In some embodiments, the repositioning device comprises an anchoring element for anchoring to a tissue wall and a band for engaging one or more papillary muscles. The band may be a device for remodeling the papillary muscles. In some embodiments, a guidewire may be inserted into the ventricle to facilitate delivery of the band.

In certain embodiments, the one or more connected elements may be inserted at different stages. In one use case, the band may be inserted before the anchoring element. For example, a first end of the band may be inserted through a catheter into the ventricle and, after encircling one or more papillary muscles, may be reinserted into the catheter such that the first end and a second end of the band are accessible to a surgeon. The anchoring element may then be inserted over the first and second ends of the band and may be delivered to a desired anchoring point in the heart (e.g., the septum or the apex). In another use case, the anchoring element may be delivered and one or more ends of the band may be attached to the anchoring element.

In some embodiments, the band may comprise an attachment device. The attachment device may comprise one or more of a hook, loop, clasp, magnet, peg, or other mechanism. In some embodiments, the attachment device may be a loop situated at or near a first end of the band that is sized to be able to fit around a second end of the band such that the second end may be passed through the loop.

In certain embodiments, at least a portion of the repositioning device may be passed through a tissue wall into a different ventricle. In one use case, the repositioning device may be inserted into right ventricle and the band may be passed through the septum or other tissue wall and into the left ventricle to allow the band to engage papillary muscles in the left ventricle. The anchoring element may be anchored to the septum or other tissue wall. In another use case, the repositioning device may be inserted into the left ventricle and the anchoring element may be passed through the septum, apex region, or other portion of tissue and anchored to a tissue wall in the right ventricle, another chamber, or outside the heart.

The anchoring element may be any kind of mechanical device configured to attach or otherwise connect to a tissue wall. For example, the anchoring element may comprise a Nitinol wire and/or mesh that may be shape set in a pre-defined shape (e.g., a ring with a slot). The anchoring element may be compressed to pass through a catheter and, after passing through the catheter, may reshape to the pre-defined shape. In some embodiments, the anchoring element may further comprise a cloth or similar material for attaching to the band. The anchoring element may be configured to lay substantially flat against the tissue wall.

The repositioning device may be positioned to cause repositioning of one or more papillary muscles while avoiding damage to the chordae tendinae. For example, the anchoring element may be positioned at a point on the septum that is lower than a top portion of the papillary muscles (e.g., where the chordae tendinae meet the papillary muscles). In this way, the anchoring element may not create an upward force at the band and may prevent the band from sliding off the papillary muscles and contacting the chordae tendinae.

At step 704, the process 700 involves wrapping the band and/or a guidewire at least partially around one or more papillary muscles in the ventricle such that that the band contacts at least one of the one or more papillary muscles. In some embodiments, the band may penetrate at least one of the one or more papillary muscles.

At step 706, the process 700 involves securing one or more ends of the band at the anchoring element and/or at a midsection of the band. In some embodiments, securing an end of the band to the anchoring element may involve inserting the end of the band through a cavity in the anchoring element. In some embodiments, both ends of the band may each be secured at the anchoring element.

In some embodiments, a first end and/or a second end of the band may comprise an attachment device for use in attaching to the anchoring element and/or band. For example, the attachment device may comprise a loop configured to fit around the band. In some embodiments, after wrapping around the one or more papillary muscles, an end of the band may be passed through the attachment device and/or the attachment device may be attached to the midsection of the band to create a complete loop around the papillary muscles. The attachment device may be slideable along the midsection of the band to allow tightening of the loop by pulling an end of the band. In some embodiments, an end of the band may be tied to the midsection of the band to create a loop around the papillary muscles.

At step 708, the process 700 involves tightening the band. In some embodiments, one or more ends of the band may be accessible to a surgeon via a catheter. For example, a first end of the band may be accessible to a surgeon while a second end of the band is inserted into a patient's heart via a catheter. The second end may be inserted into a ventricle, wrapped around one or more papillary muscles, and may be re-inserted into the catheter and may also be accessible to the surgeon via the catheter. In some embodiments, tightening the band may involve pulling one or more ends of the band. The band may be tightened as necessary to cause a desired amount of papillary muscle repositioning.

At step 710, the process 700 involves locking the band in place. In some embodiments, the anchoring element may comprise one or more locking mechanisms for use in locking the ends of the band in place. For example, the anchoring element may pinch one or more ends of the band that pass through the anchoring element to prevent the band from sliding. In some embodiments, multiple ends of the band may be tied together or otherwise connected to lock the band in place. After the band is locked in place, excess length of the band may be cut off or otherwise removed.

Placement of the repositioning device may be facilitated through use of a guidewire. The guidewire may be passed out of the catheter into the ventricle and, after encircling one or more papillary muscles, may be inserted back through the catheter such that both ends of the guidewire may be accessible to a surgeon. The band may be slid over the guidewire until it encircles the papillary muscles and may be pushed back through through the insertion point. A locking element may be slid over one or more ends of the band. In some embodiments, only one end of the band may be locked at the anchoring element.

The process 700 and/or other processes, devices, and systems disclosed herein may advantageously provide mechanisms for implementing papillary muscle and/or ventricular wall repositioning using a fully transcatheter procedure on a beating heart. In certain embodiments, valve leaflets may not be substantially touched or damaged during the process 700. Furthermore, in certain embodiments, the repositioning device may be designed to be retrievable.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

What is claimed is:

1. A method for treating a heart valve, said method comprising:
delivering a repositioning device to a ventricle of a heart, the repositioning device comprising:
an anchoring element; and
a band;
anchoring the anchoring element to a first portion of cardiac tissue;
securing a first end of the band to the anchoring element;
extending a second end of the band to encircle and at least partially contact a first papillary muscle;
tightening the band to cause repositioning of at least a portion of the first papillary muscle; and
locking the band in place.

2. The method of claim 1, further comprising securing the second end of the band to the anchoring element.

3. The method of claim 2, wherein tightening the band causes repositioning of the at least a portion of the first papillary muscle towards the anchoring element.

4. The method of claim 2, wherein securing the second end of the band to the anchoring element comprises passing the second end of the band through a cavity in the anchoring element.

5. The method of claim 1, further comprising attaching the second end of the band to a midsection of the band.

6. The method of claim 5, wherein tightening the band causes repositioning of the at least a portion of the first papillary muscle towards a second papillary muscle.

7. The method of claim 5, wherein the second end of the band comprises an attachment device configured to attach to a midsection of the band.

8. The method of claim 7, wherein the attachment device comprises one or more of a loop and a hook.

9. The method of claim 5, wherein attaching the second end of the band to a midsection of the band comprises tying the second end of the band to a midsection of the band.

10. The method of claim 5, further comprising extending the second end of the band to encircle and at least partially contact a second papillary muscle.

11. The method of claim 5, wherein the second end of the band is slidably attached to the midsection of the band to allow the second end of the band to slide along the band when the band is tightened.

12. The method of claim 1, wherein the first portion of cardiac tissue is a septum of the heart.

13. The method of claim 1, wherein the first portion of cardiac tissue is an apex region of the heart.

14. The method of claim 1, wherein the first portion of cardiac tissue is a second papillary muscle.

15. The method of claim 1, wherein the band has a hollow structure that is configured to fit around a guidewire.

16. A cardiac repositioning device comprising:
an anchoring element configured to be attached to a portion of cardiac tissue; and
a band configured to:
be secured to the anchoring element; and
encircle and at least partially contact a first papillary muscle to reposition the first papillary muscle.

17. The cardiac repositioning device of claim 16, wherein the band comprises a first end and a second end, and wherein each of the first end and the second end is configured to be secured to the anchoring element.

18. The cardiac repositioning device of claim 17, wherein the band is further configured to be tightened to cause repositioning of at least a portion of the first papillary muscle towards the anchoring element.

19. The cardiac repositioning device of claim 16, wherein the band comprises a first end and a second end, and wherein the first end is configured to be secured to the anchoring element and the second end is configured to attach to a midsection of the band.

20. The cardiac repositioning device of claim 19, wherein the band is further configured to be tightened to cause repositioning of at least a portion of the first papillary muscle towards a second papillary muscle.

21. A cardiac repositioning device comprising:
a first means configured to be attached to a portion of cardiac tissue; and
a second means configured to:
attach to the first means; and
encircle and at least partially contact a first papillary muscle to reposition the first papillary muscle.

22. The cardiac repositioning device of claim 21, wherein the second means comprises a first end and a second end, and wherein each of the first end and the second end is configured to be secured to the first means.

23. The cardiac repositioning device of claim 22, wherein the second means is further configured to be tightened to cause repositioning of at least a portion of the first papillary muscle towards the first means.

24. The cardiac repositioning device of claim 21, wherein the second means comprises a first end and a second end, and wherein the first end is configured to be secured to the first means and the second end is configured to attach to the second means.

25. The cardiac repositioning device of claim 24, wherein the second means is further configured to be tightened to cause repositioning of at least a portion of the first papillary muscle towards a second papillary muscle.
